(19) United States
(12) Patent Application Publication
(10) Pub. No.: US 2025/0195447 A1
(43) Pub. Date: Jun. 19, 2025

(54) NOVEL BIONIC NANOSPHERE M@P-WI AND ITS PREPARATION METHOD AND APPLICATION

(71) Applicant: The First Affiliated Hospital of Hainan Medical University, Haikou (CN)

(72) Inventors: Shaojiang ZHENG, Haikou (CN); Yan CHEN, Haikou (CN); Ye YU, Haikou (CN); Yonghao FAN, Haikou (CN); Wenyan LU, Haikou (CN); Wei JIE, Haikou (CN); Zhenling WAN, Haikou (CN)

(73) Assignee: The First Affiliated Hospital of Hainan Medical University, Haikou (CN)

(21) Appl. No.: 18/939,594

(22) Filed: Nov. 7, 2024

(30) Foreign Application Priority Data
Dec. 13, 2023 (CN) 202311711394.7

(51) Int. Cl. A61K 9/51; A61K 31/538; A61K 41/00; A61P 35/00

(52) U.S. Cl. CPC A61K 9/5176; A61K 9/5153; A61K 9/5192; A61K 31/538; A61K 41/0052; A61P 35/00

(57) ABSTRACT

A novel biomimetic nano-sphere M@P-WI includes PLGA nanoparticles and the membrane of breast cancer EO771 cells. The PLGA nanoparticles are encapsulated within the membrane of breast cancer EO771 cells, and IR780 and DDR2 inhibitor WRG-28 are embedded in the PLGA nanoparticles. A method for preparing and applying the novel biomimetic nano-sphere M@P-WI is also provided. The method utilizes the aforementioned novel biomimetic nano-sphere M@P-WI, to trigger the conversion of light into heat energy upon near-infrared laser irradiation. This “burns” the tumor and induces specific immune responses within the body, inhibiting tumor recurrence and metastasis. Under the action of WRG-28, it promotes apoptosis of cancer-associated fibroblasts (CAF), reduces the remodeling of tumor extracellular matrix microenvironment, enhances the distribution of nanomaterials within the tumor, and restricts the invasiveness of breast cancer cells. With laser irradiation, the photosensitizer exhibits its maximum efficacy, achieving complete elimination of TNBC rich in extracellular matrix.

### CROSS REFERENCE TO THE RELATED APPLICATIONS

This application is based upon and claims priority to Chinese Patent Application No. 202311711394.7, filed on Dec. 13, 2023, the entire contents of which are incorporated herein by reference.

### TECHNICAL FIELD

The present invention belongs to the field of tumor treatment technology and specifically relates to a novel biomimetic nano-sphere M@P-WI, including its preparation method and application.

### BACKGROUND

With the increasing trend of breast cancer incidence towards younger populations, breast cancer has become a common disease. Triple-negative breast cancer (TNBC) accounts for 15% to 20% of global breast cancer cases. However, due to its extreme invasiveness and low response to chemotherapy, TNBC patients generally have a poor prognosis, with a mortality rate exceeding 80%. Therefore, there is an urgent need for a new treatment method to improve the cure rate of TNBC and provide new strategies to inhibit metastasis.

Photothermal therapy (PTT) is an emerging treatment modality that utilizes photosensitizers to generate sufficient heat under near-infrared (NIR) light irradiation to kill tumor cells and induce an anti-tumor immune response. Compared to traditional cancer ablation methods, PTT is a novel and rapidly developing cancer treatment approach with advantages such as low toxicity and superior spatial selectivity. In addition to directly ablating tumor cells, PTT can trigger anti-tumor immune responses and form specific immune memory, thereby inhibiting tumor recurrence and metastasis. Currently, PTT research primarily focuses on tumor cell-centered therapies. However, most solid tumors, especially TNBC, have dense and complex protective barriers within the tumor microenvironment, which hinder the delivery and distribution of photosensitizers, leading to limited photothermal conversion efficiency and lack of precise targeted therapy, thus affecting the effectiveness of PTT.

Cancer-associated fibroblasts (CAFs) are one of the most abundant stromal components in the tumor microenvironment, playing a crucial role in tumor occurrence, development, metastasis, chemotherapy resistance, and immune evasion by producing various extracellular matrix (ECM) proteins and regulatory molecules. CAFs can establish and remodel ECM structure, forming a dense physical barrier that severely hinders the delivery of photosensitizers in PTT, thus weakening its efficacy. Therefore, regulating CAFs may be a promising strategy to enhance the therapeutic effectiveness of PTT in cancer treatment.

Discoidin domain receptor tyrosine kinase 2 (DDR2) is a member of the receptor tyrosine kinase (RTK) family. Current research has found high expression of DDR2 in breast cancer cells and stromal cells surrounding tumors, while normal breast epithelial cells do not express DDR2.In the tumor stroma, DDR2 expressed by CAFs can be activated by collagen proteins, promoting aggressive remodeling of collagen tissues within the tumor matrix ECM. This stimulation further enhances tumor invasiveness through a positive feedback loop between DDR2 and CAFs. WRG-28 is a potent selective inhibitor of DDR2 that blocks the interaction between DDR2 and collagen fibers by targeting the extracellular domain (ECD) of RTKs through conformational changes. It inhibits DDR2 activity, disrupts the positive feedback loop between DDR2 and CAFs, promotes CAF apoptosis, “softens” the tumor structure, improves the distribution and retention of photosensitizers, and also inhibits the invasion and migration ability of tumor cells, rendering them vulnerable to impending photothermal ablation.

Poly(lactic-co-glycolic acid) (PLGA) is a biodegradable material with excellent stability and biocompatibility, making it one of the best choices for intelligent drug delivery systems (DDS). In tumors, the aggregation of cancer cells is often attributed to adhesion molecules expressed on the cancer cell membrane, which have homologous binding domains and anchor themselves to each other through receptor-ligand interactions. In other words, the cancer cell membrane has the ability to bind homologously and home to the tumor. Additionally, due to antigen retention, nanomaterials coated with the cancer cell membrane can evade immune clearance by the reticuloendothelial system (RES). Therefore, nanospheres coated with the cancer cell membrane are expected to be ideal drug carriers.

### SUMMARY

The objective of this invention is to provide a novel biomimetic nano-sphere M@P-WI that can trigger the conversion of light into heat energy, “burn” tumors, and induce specific immune responses within the body to inhibit tumor recurrence and metastasis. Additionally, under the action of WRG-28, it promotes CAF apoptosis, reduces remodeling of the tumor extracellular matrix microenvironment, improves the distribution of nanomaterials within the tumor, and restricts the invasiveness of breast cancer cells. Ultimately, under laser irradiation, the photosensitizer exhibits its maximum efficacy, achieving complete eradication of TNBC rich in extracellular matrix.

To achieve the above objectives, this invention provides a novel biomimetic nano-sphere M@P-WI, including PLGA nanoparticles and the membrane of breast cancer E0771 cells. The PLGA nanoparticles are encapsulated within the membrane of breast cancer E0771 cells, and IR780 and DDR2 inhibitor WRG-28 are embedded in the PLGA nanoparticles.

Preferably, IR780 is an iodide compound with photothermal effect, WRG-28 is a selective inhibitor of DDR2, and PLGA is a polymer capable of loading hydrophilic or hydrophobic drugs. The encapsulation of PLGA nanoparticles within the membrane of breast cancer E0771 cells provides the PLGA nanoparticles with homologous targeting.

The method for preparing the novel biomimetic nano-sphere M@P-WI includes the following steps:
(1) Preparation of PLGA nanoparticles loaded with IR780 and WRG-28, denoted as P-WI.
(2) Extraction and purification of the membrane from breast cancer E0771 cells.
(3) Construction of the novel biomimetic nano-sphere M@P-WI with the membrane of breast cancer EO771 cells as the coating.

Preferably, in step (1), IR780 and PLGA are dissolved in dichloromethane, followed by adding WRG-28. Then, the first ultrasonic emulsification is performed under ice bath conditions, followed by the addition of Polyvinyl Alcohol (PVA) and the second ultrasonic emulsification. Isopropanol is added, and the mixture is magnetic stirred at 4° C. in the dark for 6 hours. Finally, the precipitated P-WI nanoparticles collected by centrifugation are washed three times with phosphate-buffered saline (PBS) and stored at 4° C. for future use.

Preferably, the concentration of WRG-28 is 5 mg/mL, and the mass ratio of lactic acid to glycolic acid in PLGA is 50:50, with a molecular weight of 12,000 Da Mw.

Preferably, in step (2), the Beyotime cell membrane extraction kit P0033 is used. Cultured breast cancer E0771 cells are scraped using a cell scraper, then centrifuged at 4° C., 600 g for 5 minutes to collect cells. The cells are washed again with an appropriate amount of PBS, and a small portion of the cells is counted, with a count result of not less than 1.5×10⁸ cells. Then, the cells are resuspended in 1 mL of reagent A containing Phenylmethylsulfonyl Fluoride (PMSF), followed by freeze-thawing the samples three times alternating between liquid nitrogen and room temperature. Finally, cell membrane fragments are obtained by centrifugation at 4° C., 14,000 g for 30 minutes.

Preferably, in step (3), the obtained breast cancer E0771 cell membrane is mixed with 1 mg of P-WI nanoparticles using the ultrasonic mixing method. The mixture is sonicated for 2 minutes under ice bath conditions using a 40 W ultrasonic device, followed by magnetic stirring at 4° C. for 12 hours. Finally, the mixture is washed twice to obtain the novel biomimetic nano-sphere M@P-WI.

The application of the novel biomimetic nano-sphere M@P-WI described above is specifically applied in functional testing for the treatment of triple-negative breast cancer (TNBC). The functional testing includes photothermal response detection, cytotoxicity evaluation, targeting ability assessment, promotion of CAF apoptosis, inhibition of collagen fiber deposition, tumor-killing effect, enhancement of immune response, and suppression of tumor metastasis.

The advantages and positive effects of the novel biomimetic nano-sphere M@P-WI, its preparation method, and application provided by this invention are as follows:

The novel biomimetic nano-sphere M@P-WI prepared in this invention has advantages such as non-invasiveness, strong specificity, high stability, excellent biocompatibility, low toxicity, low cost, and easy preparation.

By combining PTT with the action of WRG-28 to inhibit DDR2 activity, this invention not only promotes CAF apoptosis and reduces remodeling of the tumor extracellular matrix microenvironment, improving the distribution of nanomaterials within the tumor but also restricts the invasiveness of breast cancer cells. Ultimately, under laser irradiation, the photosensitizer exhibits its maximum efficacy, achieving complete eradication of TNBC rich in extracellular matrix while stimulating sufficient immune responses within the body to resist tumor recurrence and inhibit metastasis.

The novel biomimetic nano-sphere M@P-WI provided by this invention has broad prospects in efficiently eliminating tumors, inhibiting tumor metastasis and recurrence in cancer treatment.

The technical solution of this invention will be further described in detail through the attached drawings and exemplary embodiments.

### BRIEF DESCRIPTION OF THE DRAWINGS

FIG. 1: Schematic diagram of the construction of the bionic nanosphere M@P-WI in the present invention.

FIG. 2: Transmission electron microscopy image of P-WI nanospheres in Embodiment 1 of the present invention.

FIG. 3: Transmission electron microscopy image of the bionic nanosphere M@P-WI in Embodiment 1 of the present invention.

FIG. 4: SDS-PAGE analysis of protein composition from EO771 cell membranes, M@P-WI, and P-WI in Embodiment 1 of the present invention.

FIG. 5: In vitro near-infrared laser thermal effect of M@P-WI at different concentrations in Embodiment 2 of the present invention.

FIG. 6: In vitro homologous targeting capability of M@P-WI in Embodiment 3 of the present invention.

FIG. 7: In vivo homologous targeting capability of M@P-WI in Embodiment 3 of the present invention.

FIG. 8: Flow cytometry results of CAF cell apoptosis in Embodiment 5 of the present invention.

FIG. 9: Collagen fiber content of CAF stained with Sirius Red in Embodiment 6 of the present invention.

FIG. 10: Evaluation of tumor tissue hardness before and after treatment in Embodiment 7 of the present invention.

FIG. 11: Distribution of nanomaterials within the tumor in each group in Embodiment 8 of the present invention.

FIG. 12: Change in tumor volume of mice during in vivo treatment with M@P-WI in Embodiment 9 of the present invention.

FIG. 13: Weight changes of EO771 tumor-bearing mice during in vivo treatment in Embodiment 9 of the present invention.

FIG. 14: Comparison of ex vivo tumors from EO771 tumor-bearing mice after the end of in vivo treatment in Embodiment 9 of the present invention.

FIG. 15: Blood routine tests and biochemical indicators of E0771 tumor-bearing mice after the end of in vivo treatment in Embodiment 9 of the present invention.

FIG. 16: HE staining of ex vivo organs from EO771 tumor-bearing mice after the end of in vivo treatment in Embodiment 9 of the present invention.

FIGS. 17A-17H: Effects of M@P-WI treatment on immune response in vivo in Embodiment 10 of the present invention.

FIG. 18: Schematic diagram of the treatment process of M@P-WI inhibiting distant tumor metastasis in Embodiment 11 of the present invention.

FIG. 19: Live imaging of lung metastasis in mice from each treatment group.

### DETAILED DESCRIPTION OF THE EMBODIMENTS

The following provides a further explanation of the technical solution of the present invention through accompanying diagrams and implementation examples. Unless otherwise defined, the technical or scientific terms used in the present invention shall have their generally accepted meanings as understood by those skilled in the relevant field of the invention.

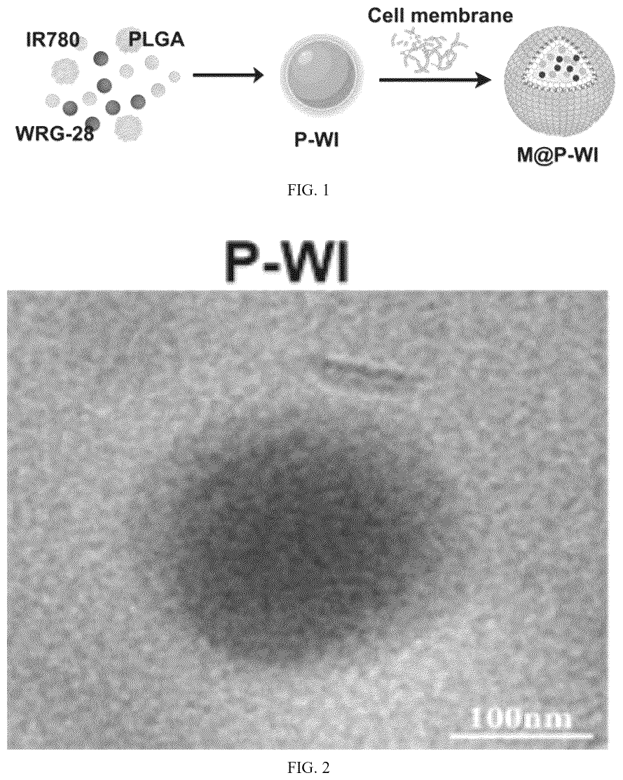

As shown in FIG. 1, a novel bionic nanosphere M@P-WI includes PLGA nanoparticles (P-WI) and the cell membrane derived from breast cancer E0771 cells. The PLGA nanoparticles (P-WI) are encapsulated within the breast cancer E0771 cell membrane, and they contain IR780 and the DDR2 inhibitor WRG-28.

IR780 is an iodide with photothermal effects that can convert light into heat upon near-infrared laser irradiation, effectively “burning” tumors while simultaneously inducing a specific immune response in the body to inhibit tumor recurrence and metastasis. WRG-28 is a potent selective inhibitor of DDR2, which suppresses DDR2 activity by blocking its interaction with collagen fibers, thereby promoting the apoptosis of cancer-associated fibroblasts (CAFs) and limiting the invasiveness of breast cancer cells. PLGA is a polymer known for its excellent stability, biodegradability, and good biocompatibility, making it suitable for loading both hydrophilic and hydrophobic drugs. The encapsulation of the breast cancer EO771 cell membrane provides P-WI with homologous targeting capabilities, ultimately resulting in the composite M@P-WI, which is designed for personalized and precise therapy.

The preparation method for the novel bionic nanosphere M@P-WI includes the following steps:

### Preparation of PLGA Nanoparticles P-WI Loaded with IR780 and WRG-28

The oil-in-water emulsion synthesis method is employed. First, 2 mg of IR 780 and 50 mg of PLGA (molecular weight 12,000 Da) are completely dissolved in 2 mL of chloroform (CHCl₃). Then, 200 μL of WRG-28 at a concentration of 5 mg/mL is added. If fluorescently labeled M@P-WI nanospheres are to be prepared, Dil fluorescent dye can also be dissolved in the chloroform during this step. Next, ultrasonic emulsification is carried out in an ice bath (45 W, 3 min, 5″ on, 5″ off). Following this, 6 mL of PVA (4% w/v) is added, and secondary ultrasonic emulsification is performed (45 W, 3 min, 5″ on, 5″ off). Subsequently, 6 mL of isopropanol (2% w/v) is added, and the mixture is magnetically stirred in the dark at 4° C. for 6 hours to remove the organic solvent and uniformly disperse the nanoparticles. Afterward, the P-WI nanosphere precipitate is collected by centrifugation at 16,000 g for 25 minutes at 4° C., washed three times with PBS, and stored at 4° C. for later use. The mass ratio of lactic acid to glycolic acid in PLGA is 50:50.

### Extraction and Purification of Breast Cancer E0771 Cell Membrane

The cell membrane extraction kit (Beyotime, P0033) is used for this procedure. When the EO771 cells reach 90%-95% confluency, they are collected. First, wash the cells once with PBS. Using a cell scraper under ice bath conditions, scrape off the cells, and then centrifuge at 600 g for 5 minutes at 4° C. to collect the cells. Add an appropriate amount of PBS and repeat the centrifugation step to wash the cells, reserving a small amount for counting. The total number of collected cells should not be less than 1.5×10⁸ cells. Next, add 1 mL of reagent A containing PMSF and gently resuspend the cells. Then, subject the sample to three freeze-thaw cycles using liquid nitrogen and room temperature. Finally, centrifuge at 14,000 g for 30 minutes at 4° C. to obtain the precipitate of cell membrane fragments.

### Construction of Novel Bionic Nanosphere M@P-WI Coated with Breast Cancer E0771 Cell Membrane

Mix 1 mg of the EO771 cell membrane obtained in step (2) with the P-WI nanospheres prepared in step (1). Under ice bath conditions, treat the mixture with ultrasound at 40 W for 2 minutes. Then, magnetically stir the mixture at 4° C. for 12 hours. After that, wash the resulting solution with PBS twice to obtain the M@P-WI nanospheres.

The principle of precise targeted therapy in this invention is based on the M@P-WI nanospheres, which consist of the photothermal iodide IR780 and the DDR2 inhibitor WRG-28, both encapsulated within a biodegradable polymer PLGA. Additionally, the concept of homologous targeting is utilized; that is, the aggregation of multiple cancer cells in tumors is often attributed to the expression of surface adhesion molecules on the cancer cell membrane, which possess homologous adhesive domains. Examples of these adhesion molecules include epithelial cell adhesion molecule (EpCAM) and galectin-3. The cancer cell membrane demonstrates the ability to bind specifically with homologous cancer cells. Therefore, nanocarriers coated with cancer cell membranes exhibit targeted binding properties specific to cancer cells.

The application of the novel bionic nanosphere M@P-WI is focused on functional testing in the treatment of triple-negative breast cancer. The functional assays include:

Photothermal response detection, cytotoxicity assessment, targeting capability, promotion of CAF apoptosis, inhibition of collagen fiber deposition, tumor killing effect, enhancement of immune response and inhibition of tumor metastasis.

The main reagents and materials are as follows:

PLGA (DLLA: GA =50:50, PLGA 12,000 Da Mw) purchased from Shandong Jinan Daigang Biotechnology Co., Ltd. IR780 (Sigma-Aldrich, 425311). WRG-28 (MedChemExpress, HY-114169). DiI Cell Membrane Red Fluorescent Probe, Penicillin-Streptomycin Solution, Trypsin, Membrane Protein Extraction Kit, Phenylmethylsulfonyl Fluoride (PMSF), and CCK-8 obtained from Beyotime. Chloroform (CHCl₃), Polyvinyl Alcohol (PVA, 25,000 Mw), and Isopropanol purchased from China National Pharmaceutical Group Chemical Reagents Co., Ltd. Sirius Red Staining Kit (Solarbio, G1472), Masson's Trichrome Staining Kit (Solarbio, G1340), and Enzyme-Linked Immunosorbent Assay (ELISA) Kits for mouse IL-6, IL-12, TNF-α, and IL-10 obtained from Enzyme Industry Co., Ltd. Copper Grids, Centrifuge Tubes, Pipettes and Tips, Permanent Markers, and Cell Culture Plates.

### Implementation Example 1

In this implementation example, the novel bionic nanosphere M@P-WI is characterized and tested to verify the feasibility of the technical scheme constructed in this invention.

### Observation of Nanosphere Morphology

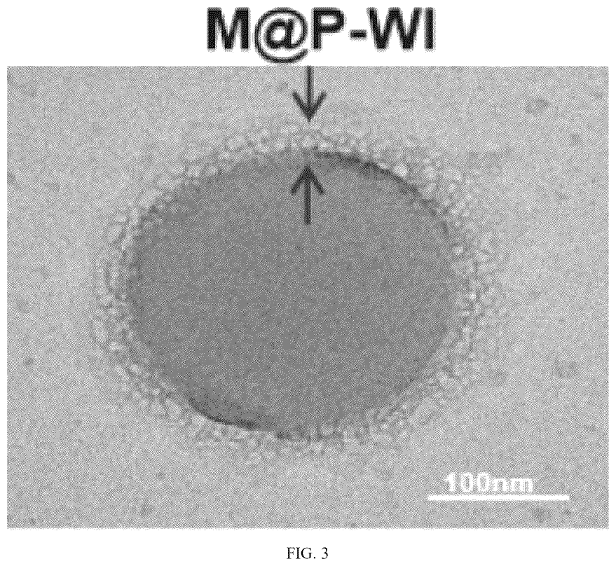

To observe the morphology of the nanospheres, 10 μL of the P-WI solution was diluted 20 times with PBS and then dropped onto a copper grid laid on filter paper. After drying overnight at 4° C., transmission electron microscopy (TEM) scanning was performed to obtain the TEM morphology images, as shown in FIG. 2. The results indicate that the P-WI particles are uniform in size, with a diameter of approximately 260 nm and good dispersion.

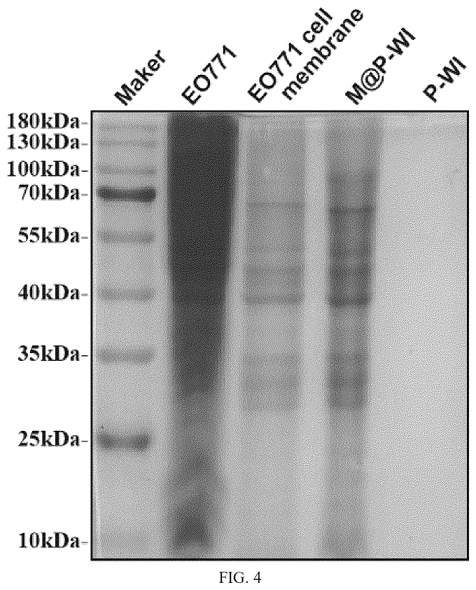

After further ultrasonic mixing of P-WI with the EO771 cell membrane, its TEM morphology was observed, as shown in FIG. 3. It can be seen that the outer surface of the P-WI spheres is enveloped by a continuous and intact layer, increasing the diameter to about 300 nm. This indicates that the cell membrane, fragmented by ultrasound, successfully self-assembled and wrapped around the P-WI nanospheres while still maintaining high dispersion.

### Analysis of Protein Band Composition

To further confirm that the breast cancer EO771 cell membrane successfully coated the surface of the P-WI nanospheres, Coomassie Brilliant Blue staining SDS-PAGE gel analysis was performed on the protein band compositions of the breast cancer EO771 cell membrane, M@P-WI nanospheres, and P-WI nanospheres. Through this method, different samples' protein bands can be clearly observed, confirming that the cell membrane has successfully covered the surface of the nanospheres.

The results of this experiment will help further evaluate the potential application of M@P-WI nanospheres in treating triple-negative breast cancer, including the following steps:

First, prepare a 10% electrophoresis gel according to the instructions of the SDS-PAGE gel kit and soak it in the electrophoresis buffer for later use. Then, take 10 μL of E0771 cell membrane, M@P-WI nanospheres, and P-WI nanospheres, respectively, dilute them with Loading Buffer, and heat in a metal bath at 100° C. for 10 minutes to denature the proteins. After heating, add 10 μL of the above samples to each well of the SDS-PAGE gel, and then adjust the electrophoresis apparatus to run at 100V until the stacking gel is completed, followed by running at 120V until the end of the process.

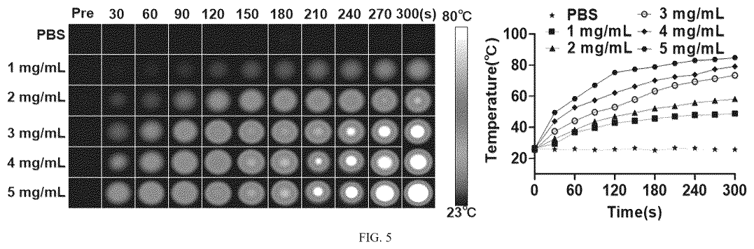

After electrophoresis, cut out the gel containing the Marker and samples, and add a sufficient amount of Coomassie Brilliant Blue staining solution to completely cover the gel, shaking gently on a horizontal shaker for 1 hour. After staining, add an appropriate amount of Coomassie Brilliant Blue destaining solution and shake gently on a horizontal shaker for 6 hours. During this time, replace the destaining solution 3-6 times until almost all the blue background is removed. The results are shown in FIG. 4, indicating that the M@P-WI nanospheres have a protein composition almost identical to that of the original E0771 cell membrane.

### Implementation Example 2

To evaluate the near-infrared photothermal response characteristics of the novel bionic nanosphere M@P-WI, the steps are as follows:

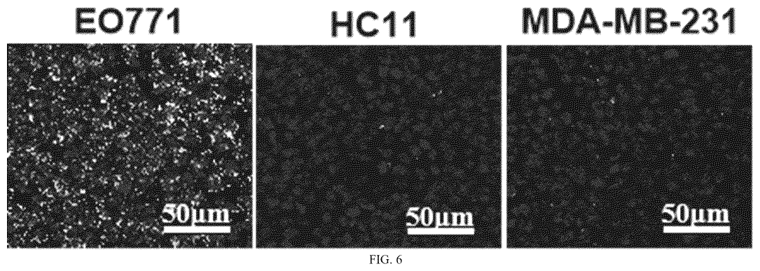

First, assess the photothermal effect of M@P-WI at different concentrations (1, 2, 3, 4, and 5 mg/mL) using an 808 nm near-infrared laser. Every 30 seconds, use an infrared thermal imaging camera to record the temperature changes, observing for a total duration of 5 minutes. The progression of the photothermal effect strength is shown in FIG. 5. As seen from FIG. 5, the in vitro photothermal effect of M@P-WI has a significant concentration dependence.

### Implementation Example 3

To evaluate the homogenous targeting ability of the novel bionic nanoparticles M@P-WI, the steps are as follows:

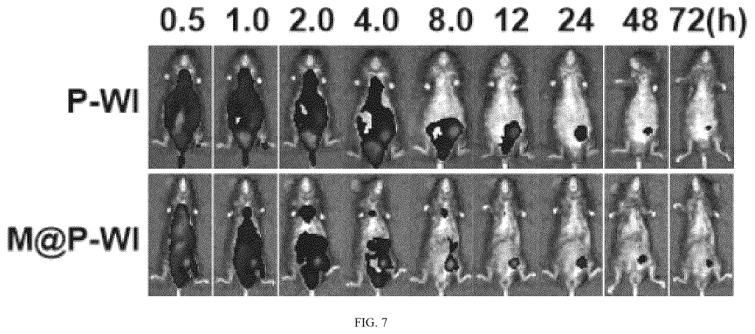

E0771 cells, MDA-MB-231 cells, and CH11 cells were respectively seeded in confocal culture dishes and cultured for 24 hours. Then, DiI-labeled M@P-WI nanoparticles (equivalent PLGA concentration of 50 g/mL) were added and co-cultured for 2 hours. Afterward, DAPI was used to stain the cell nuclei, and observations were made using a confocal laser microscope. The results, as shown in FIG. 6, indicate that the EO771 group had higher uptake efficiency and fluorescence intensity, proving that M@P-WI has a significant specific binding ability with homologous E0771 cells.

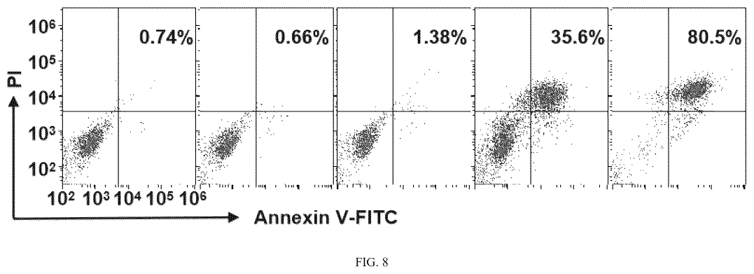

Based on the above in vitro results, we further validated the targeting ability of M@P-WI in vivo using EO771 tumor-bearing mice. The tumor-bearing mice were randomly divided into two groups (n=5), namely the M@P-WI group and the P-WI group. The mice were intravenously injected via the tail with 200 μL of M@P-WI or P-WI nanoparticles at an equivalent PLGA concentration of 2 mg/mL. The in vivo distribution and metabolism of the nanoparticles were observed at different time points after injection (0.5 h, 1 h, 2 h, 4 h, 8 h, 12 h, 24 h, 48 h, and 72 h) using small animal live imaging. The results, as shown in FIG. 7, indicated that with the passage of time, the fluorescence signals of the M@P-WI group significantly accumulated at the tumor site of the mice and peaked at 12 hours. Moreover, significant fluorescence signals were still clearly visible 72 hours post-injection, indicating that M@P-WI nanoparticles can stably reside at the tumor site.

### Implementation Example 4

To evaluate the ability of the novel bionic nanoparticles M@P-WI to promote CAF cell apoptosis, the steps are as follows:

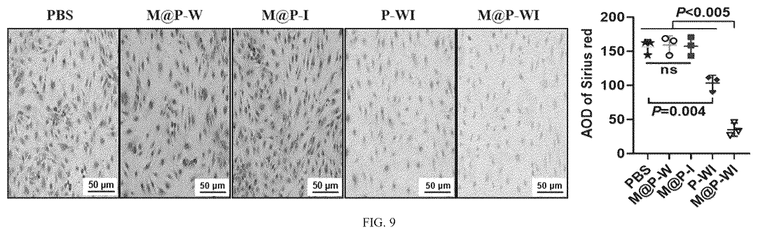

First, EO771 cells (8×10\hat{ }4 per well) were cultured in a 6-well plate overnight. Then, an equal amount of M@P-WI at a PLGA concentration of 400 μg/mL was added and co-cultured for 4 hours. The control group received an equal amount of PBS, M@P-W, P-WI or M@P-I. Subsequently, the samples were irradiated with 808 nm near-infrared light at a power density of 1.5 W/cm² for 5 minutes. Following this, each group's culture medium was co-cultured with CAF for 24 hours. Finally, apoptosis was analyzed using Annexin V-FITC apoptosis detection kit combined with flow cytometry. The results, shown in FIG. 8, indicate that the CAF apoptosis in the M@P-WI group significantly increased, whereas the M@P-I group had no significant effect on CAF viability due to the lack of WRG-28. This result demonstrates that M@P-WI can promote CAF apoptosis.

### Implementation Example 5

To assess the ability of bionic nanoparticles M@P-WI to inhibit CAF collagen fiber generation, the steps are as follows:

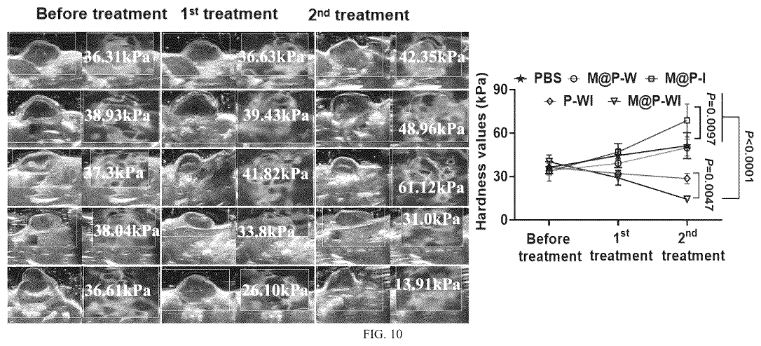

First, EO771 cells (8×10\hat{ }4 per well) were cultured in a 6-well plate overnight. An equal amount of M@P-WI at a PLGA concentration of 400 μg/mL was added and co-cultured for 4 hours. The control group received an equal amount of PBS, M@P-W, P-WI or M@P-I. Following this, the samples were irradiated with 808 nm near-infrared light at a power density of 1.5 W/cm² for 5 minutes. Each group's culture medium was then co-cultured with CAF for 24 hours. Lastly, the collagen fiber content in CAF was analyzed using Sirius Red staining kit. The results, shown in FIG. 9, indicate that the M@P-WI group significantly inhibited collagen fiber formation.

### Implementation Example 6

To evaluate the effect of bionic nanoparticles M@P-WI on tumor tissue softening, the steps are as follows:

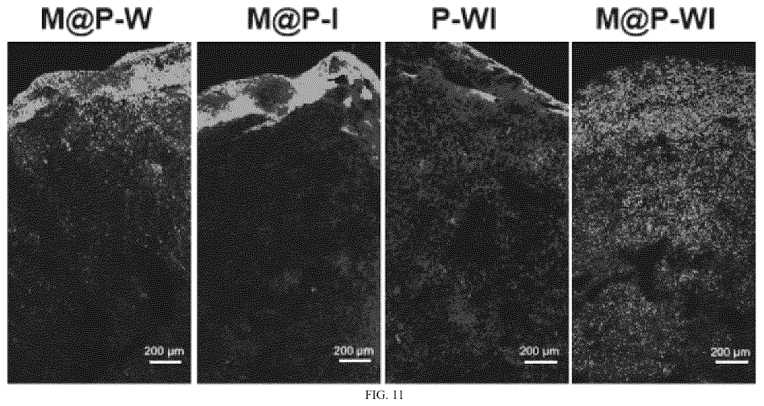

EO771 tumor-bearing mice were randomly divided into five groups (n=3): PBS, M@P-W, P-WI, M@P-I and M@P-WI groups. After intravenous injection of 200 μL of the corresponding drug for 4 hours, the mice were irradiated with 808 nm laser (1.5 W/cm², 10 min). After 24 hours, ultrasound elastography was used to detect the hardness of the tumor tissue. The results, shown in FIG. 10, indicate that the elastic value of tumors in the M@P-WI group decreased after treatment, while there was no significant change in the elastic value of the M@P-I group, indicating that M@P-WI can effectively reduce tumor tissue hardness.

### Implementation Example 7

To evaluate the advantages of the novel bionic nanoparticles M@P-WI in tumor distribution and retention, the steps are as follows:

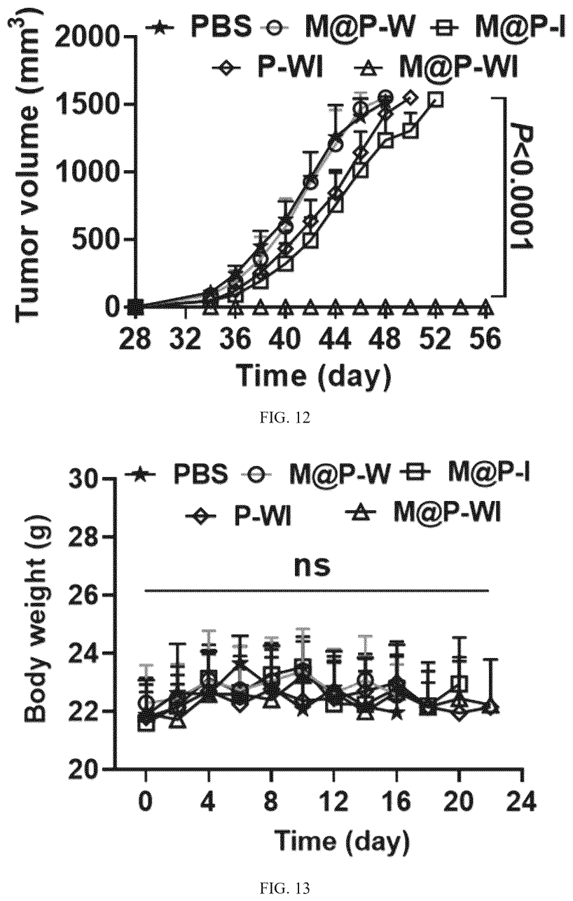

EO771 tumor-bearing mice were randomly divided into five groups (n =3): PBS, M@P-W, P-WI, M@P-I and M@P-WI groups. After intravenous injection of 200 μL of the corresponding drug for 4 hours, the mice were irradiated with 808 nm laser (1.5 W/cm², 10 min). After another 24 hours, 200 μL of the corresponding drug was injected again, and after 4 hours, the tumors were excised for fast freezing sectioning. After nuclear staining with DAPI, the fluorescence distribution of the nanoparticles was observed using a confocal microscope. The results, shown in FIG. 11, indicate that the fluorescence distribution and retention of the M@P-WI group inside the tumor were significantly better than those of the M@P-I group.

### Implementation Example 8

To evaluate the anti-tumor capability of the novel bionic nanoparticles M@P-WI, the steps are as follows:

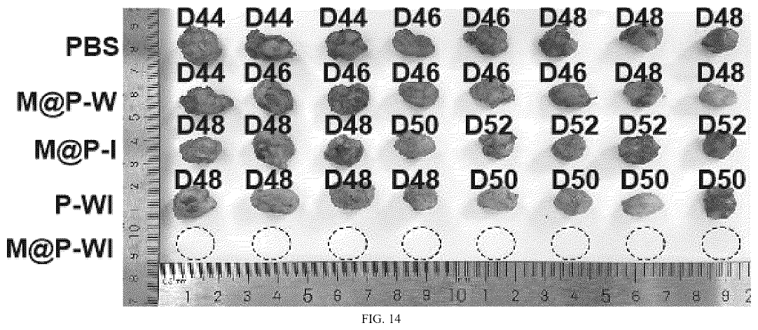

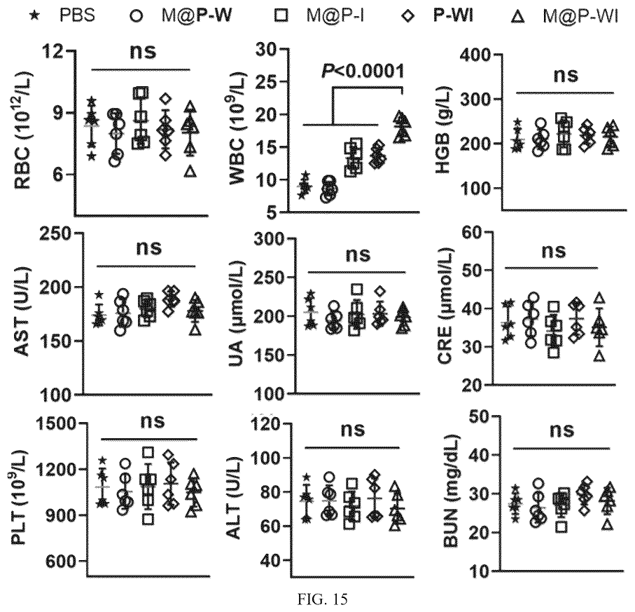

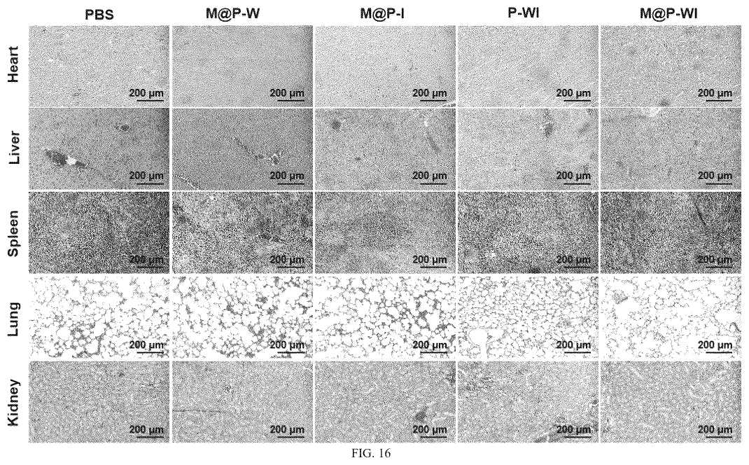

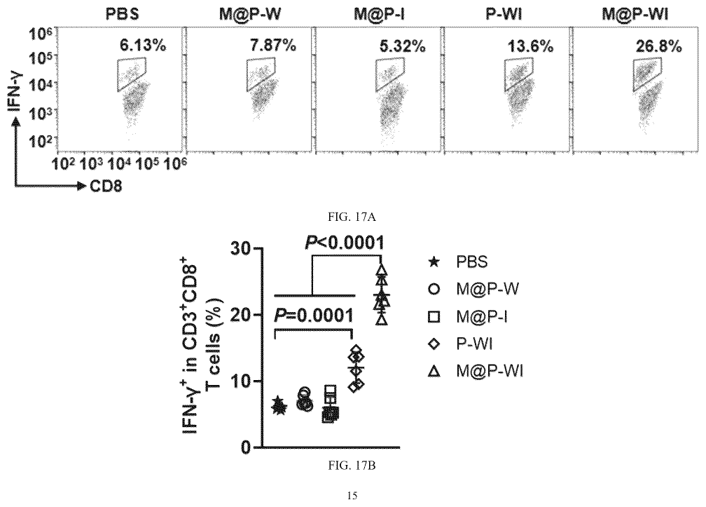

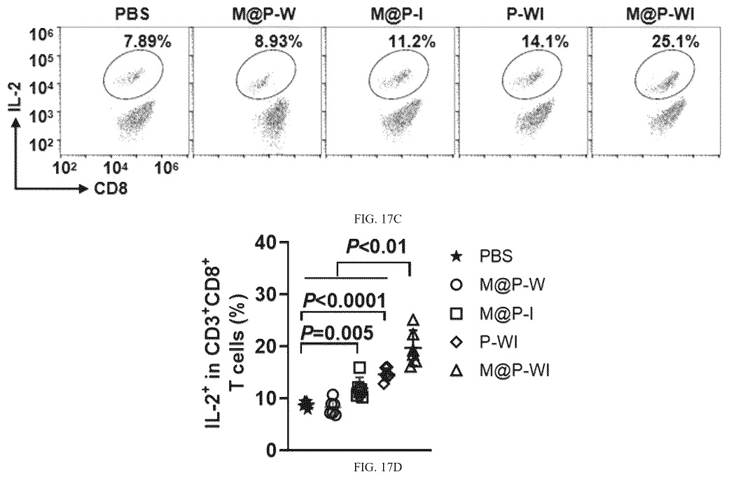

Tumor-bearing mice were randomly divided into three groups (n=8): PBS, M@P-W, P-WI, M@P-I and M@P-WI groups. The mice received a tail vein injection of 200 μL of M@P-WI nanoparticles at a concentration of 2 mg/mL, while the control group received an equal dose of PBS or M@P-I. Four hours post-injection, the tumor site was irradiated with 808 nm near-infrared laser at a power density of 1.5 W/cm² for 10 minutes, with treatments repeated every 3 days for a total of 3 times. During the treatment period, tumor volume and mouse weight changes were recorded every 3 days. The results, shown in FIG. 12, indicate that the tumor volume in the M@P-WI group gradually decreased, with most tumors eliminated by the end of the treatment period. Additionally, there was minimal weight change in the mice during treatment (as shown in FIG. 13). During the treatment period, if the tumor volume of the mice exceeds 1500 mm³, it is considered as death, and the tumor tissue is excised. If the volume has not reached 1500 mm³, treatment will continue until the day after the end of the treatment course, at which point the mice will be euthanized, and the tumor will be excised. The results, shown in FIG. 14, demonstrated that the ex vivo tumors indicated significant therapeutic effects in the M@P-WI group. Blood routine tests, biochemical indicators (as shown in FIG. 15), and HE staining of ex vivo organs (as shown in FIG. 16) also indicated high biosafety of the M@P-WI treatment.

### Implementation Example 9

To evaluate the immune response-inducing ability of the novel bionic nanoparticles M@P-WI, the steps are as follows:

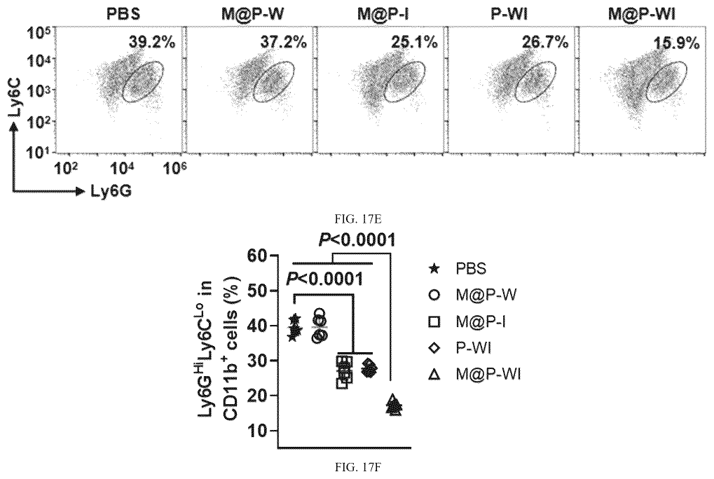

The EO771 tumor-bearing mice were randomly divided into three groups and received the same in vivo treatment as described in Embodiment 6. On the second day after the end of the treatment course, CD8⁺ T lymphocytes were collected from the tumors of mice in different groups, and flow cytometry was used to analyze the levels of CD8⁺ IFN-γ⁺ T cells, CD8⁺ IL-2⁺ T cells, PMN-MDSCs, and M2-phenotype macrophages. The results are shown in FIGS. 17A-17H. The levels of CD8⁺ IFN-γ⁺ T cells and CD8+IL-2⁺ T cells in the M@P-WI group were significantly higher than those in the control group, while the levels of immunosuppressive PMN-MDSCs and M2-phenotype macrophages were significantly lower than those in the other groups. This indicates that M@P-WI has a notable ability to induce anti-tumor immune function.

### Implementation Example 10

To evaluate the ability of the novel bionic nanoparticles M@P-WI to inhibit distant metastasis of breast cancer, the steps are as follows:

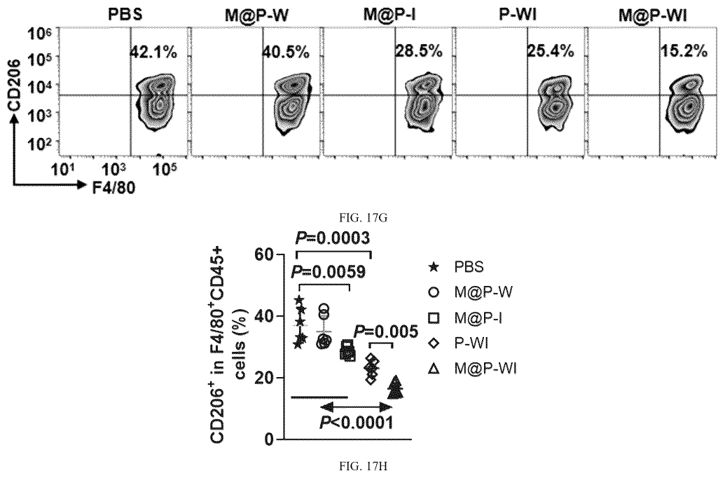

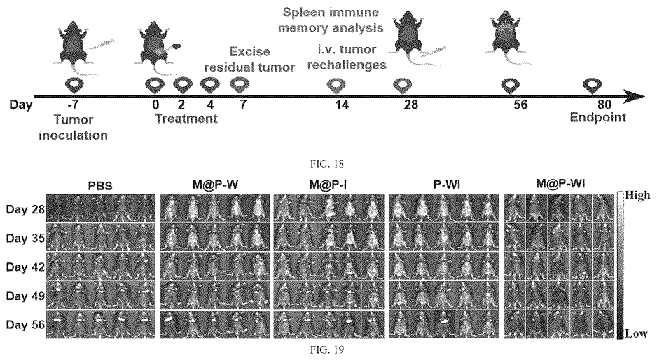

To establish a mouse tumor recurrence model, C57BL/6 mice were implanted with E0771 tumors rich in stroma, with an initial tumor volume of approximately 100 mm³. The mice were treated with PBS, M@P-Ws, M@P-Is, P-WIs, or M@P-WIs, followed by near-infrared laser irradiation. Three days after treatment (on day 7), tumors in the PBS group, M@P-W group, M@P-I group, and P-WI group were surgically excised; however, all primary tumors in the M@P-WI group had disappeared, eliminating the need for surgery. On day 28, 5×10\hat{ }5 Luci-E0771 cells were injected via the tail vein into the mice from each treatment group to establish a lung metastasis model. The experimental procedure is shown in FIG. 18. Live imaging was conducted at different time points to observe the growth of lung tumors in each group. The results are shown in FIG. 19. Except for the M@P-WI group, significant lung tumors were observed in all other treatment groups, indicating that treatment with M@P-WI can resist distant tumor metastasis.

Therefore, the present invention utilizes a novel bionic nanosphere M@P-WI, which can convert light into heat energy upon near-infrared laser irradiation to “burn” tumors and induce specific immune responses in the body, thereby inhibiting tumor recurrence and metastasis. At the same time, under the action of WRG-28, it promotes CAF apoptosis and reduces the remodeling of the tumor extracellular matrix microenvironment, thereby enhancing the distribution of the nanomaterials within the tumor. Additionally, it limits the invasiveness of breast cancer cells. Ultimately, under laser irradiation, the photosensitizer exerts its maximum efficacy, achieving a complete ablation of TNBC rich in extracellular matrix.

It should be noted that the above embodiments are intended to illustrate the technical solutions of the present invention and not to limit them. Although the present invention has been described in detail with reference to preferred embodiments, those skilled in the art should understand that modifications or equivalent substitutions can still be made to the technical solutions of the present invention without departing from the spirit and scope of the invention.

What is claimed is:

1. A biomimetic nano-sphere M@P-WI, comprising poly (lactic-co-glycolic acid) (PLGA) nanoparticles and a membrane of breast cancer E0771 cells, wherein the PLGA nanoparticles are encapsulated within the membrane of the breast cancer EO771 cells, and an IR780 and a DDR2 inhibitor WRG-28 are embedded in the PLGA nanoparticles.

2. The biomimetic nano-sphere M@P-WI according to claim 1, wherein the IR780 is an iodide compound with a photothermal effect, the DDR2 inhibitor WRG-28 is a selective inhibitor of a DDR2, and a PLGA is a polymer configured for loading hydrophilic or hydrophobic drugs; and an encapsulation of the PLGA nanoparticles within the membrane of the breast cancer E0771 cells provides the PLGA nanoparticles with a homologous targeting.

3. A method for preparing the biomimetic nano-sphere M@P-WI according to claim 1, comprising the following steps: (1) preparing PLGA nanoparticles loaded with the IR780 and the DDR2 inhibitor WRG-28, wherein the PLGA nanoparticles loaded with the IR780 and the DDR2 inhibitor WRG-28 are denoted as P-WI nanoparticles; (2) extracting and purifying the membrane of the breast cancer EO771 cells; and (3) constructing the biomimetic nano-sphere M@P-WI with the membrane of the breast cancer EO771 cells as a coating.

4. The method according to claim 3, wherein the step (1) comprises: dissolving the IR780 and the PLGA nanoparticles in dichloromethane, adding the DDR2 inhibitor WRG-28, then performing a first ultrasonic emulsification under ice bath conditions, followed by an addition of polyvinyl alcohol (PVA) and a second ultrasonic emulsification; and subsequently, adding isopropanol and stirring magnetically in a dark at 4° C. for 6 hours, and finally collecting precipitated P-WI nanoparticles by a centrifugation and washing the precipitated P-WI nanoparticles three times with phosphate-buffered saline (PBS) before storing at 4° C. for a future use.

5. The method according to claim 4, wherein a concentration of the DDR2 inhibitor WRG-28 is 5 mg/mL, a mass ratio of lactic acid to glycolic acid in the PLGA nanoparticles is 50:50, and a molecular weight of the PLGA nanoparticles is 12000 Da Mw.

6. The method according to claim 3, wherein the step (2) comprises: using a cell membrane extraction kit, scraping cultured breast cancer EO771 cells with a cell scraper, centrifuging collected cells at 4° C., 600 g for 5 minutes to obtain centrifuged cells, washing the centrifuged cells with an appropriate amount of PBS to obtain washed cells, and taking a small amount of the washed cells for a counting, wherein a cell count is not allowed to be less than 1.5×10⁸ cells, then, adding 1 mL of a reagent containing phenylmethylsulfonyl fluoride (PMSF) to resuspend the washed cells to obtain resuspended cells, followed by freeze-thawing the resuspended cells three times in liquid nitrogen and at a room temperature, and finally obtaining cell membrane fragments by centrifuging at 4° C., 14000 g for 30 minutes.

7. The method according to claim 3, wherein the step (3) comprises: using an ultrasonic mixing method, mixing the membrane of the breast cancer EO771 cells with 1 mg of the P-WI nanoparticles, then sonicating for 2 minutes under ice bath conditions using a 40 W ultrasonic device, followed by a magnetic stirring at 4° C. for 12 hours to obtain a mixture, and finally, washing the mixture twice to obtain the biomimetic nano-sphere M@P-WI.

8. A method for a functional testing for a treatment of a triple-negative breast cancer (TNBC), comprising using the biomimetic nano-sphere M@P-WI according to claim 1, wherein the functional testing comprises a photothermal response detection, a cytotoxicity evaluation, a targeting ability assessment, a promotion of a cancer-associated fibroblast (CAF) apoptosis, an inhibition of a collagen fiber deposition, a tumor-killing effect, an enhancement of an immune response, and a suppression of a tumor metastasis.

9. The method according to claim 3, wherein in the biomimetic nano-sphere M@P-WI, the IR780 is an iodide compound with a photothermal effect, the DDR2 inhibitor WRG-28 is a selective inhibitor of a DDR2, and a PLGA is a polymer configured for loading hydrophilic or hydrophobic drugs; and an encapsulation of the PLGA nanoparticles within the membrane of the breast cancer EO771 cells provides the PLGA nanoparticles with a homologous targeting.

10. The method according to claim 8, wherein in the biomimetic nano-sphere M@P-WI, the IR780 is an iodide compound with a photothermal effect, the DDR2 inhibitor WRG-28 is a selective inhibitor of a DDR2, and a PLGA is a polymer configured for loading hydrophilic or hydrophobic drugs; and an encapsulation of the PLGA nanoparticles within the membrane of the breast cancer EO771 cells provides the PLGA nanoparticles with a homologous targeting.
